SEQUENCE LISTING (33 sequences)

SEQ ID NO: 1 - protein, 18 aa, artificial sequence
  Synthetic peptide PP2Ac
ETVTLLVALKVRYRERIT

SEQ ID NO: 2 - protein, 22 aa, artificial sequence
  Synthetic peptide SET
PSSKSTEIKWKSGKDLTKRSSQ

SEQ ID NO: 3 - protein, 11 aa, artificial sequence
  synthetic peptide
  VARIANT (1)..(1): X is K, VK or is absent
  VARIANT (7)..(7): X is any amino acids
  VARIANT (10)..(10): X is K, KI or is absent
  VARIANT (11)..(11): X is a sequence of 1 to 4 amino acids or is absent
XKKKIKXEIXX

SEQ ID NO: 4 - protein, 18 aa, artificial sequence
  Synthetic peptide
RQKRLIRQKRLIRQKRLI

SEQ ID NO: 5 - protein, 18 aa, artificial sequence
  Synthetic peptide
RHSRIGRHSRIGRHSRIG

SEQ ID NO: 6 - protein, 16 aa, artificial sequence
  Synthetic peptide
RHSRIGIIQQRRTRNG

SEQ ID NO: 7 - protein, 16 aa, artificial sequence
  synthetic peptide
RHSRIGVTRQRRARNG

SEQ ID NO: 8 - protein, 15 aa, artificial sequence
  Synthetic peptide
RRRRRRRSRGRRRTY

SEQ ID NO: 9 - protein, 12 aa, artificial sequence
  Synthetic peptide
VKKKKIKREIKI

SEQ ID NO: 10 - protein, 12 aa, artificial sequence
  synthetic peptide
VKKKKIKAEIKI

SEQ ID NO: 11 - protein, 12 aa, artificial sequence
  Synthetic peptide
VKKKKIKKEIKI

SEQ ID NO: 12 - protein, 12 aa, artificial sequence
  Synthetic peptide
VKKKKIKNEIKI

SEQ ID NO: 13 - protein, 9 aa, artificial Sequence
  Synthetic peptide
RKKRRQRRR

SEQ ID NO: 14 - protein, 11 aa, artificial sequence
  synthetic peptide
YGRKKRRQRRR

SEQ ID NO: 15 - protein, 9 aa, artificial sequence
  Synthetic peptide
RRRRRRRRR

SEQ ID NO: 16 - protein, 11 aa, artificial sequence
  synthetic peptide
RRRRRRRRRRR

SEQ ID NO: 17 - protein, 32 aa, artificial sequence
  synthetic peptide HA2-R9
GLFEAIEGFIENGWEGMIDGWYGRRRRRRRRR

SEQ ID NO: 18 - protein, 16 aa, artificial sequence
  Synthetic peptide penetratin
RQIKIWFQNRRMKWKK

SEQ ID NO: 19 - protein, 27 aa, artificial sequence
  Synthetic peptide Transportan
GWTLNSAGYLLGKINLKALAALAKKIL

SEQ ID NO: 20 - protein, 33 aa, artificial sequence
  Synthetic peptide Maurocalcine
GDCLPHLKLCKENKDCCSKKCKRRGTNIEKRCR

SEQ ID NO: 21 - protein, 10 aa, artificial sequence
  synthetic peptide decalysine
KKKKKKKKKK

SEQ ID NO: 22 - protein, 11 aa, artificial sequence
  Synthetic peptide HIV-Tat derived PTD4
YARAAARQARA

SEQ ID NO: 23 - protein, 12 aa, artificial sequence
  synthetic peptide
PLSSIFSRIGDP

SEQ ID NO: 24 - protein, 28 aa, artificial sequence
  synthetic petide mPrP1-28
MANLGYWLLALFVTMWTDVGLCKKRPKP

SEQ ID NO: 25 - protein, 35 aa, artificial sequence
  Synthetic peptide POD
GGGARKKAAKAARKKAAKAARKKAAKAARKKAAKA

SEQ ID NO: 26 - protein, 19 aa, artificial sequence
  synthetic peptide pVEC
LLIILRRRRIRKQAHAHSK

SEQ ID NO: 27 - protein, 23 aa, artificial sequence
  Synthetic peptide EB1
LIRLWSHLIHIWFQNRRLKWKKK

SEQ ID NO: 28 - protein, 22 aa, artificial sequence
  synthetic peptide Rath
TPWWRLWTKWHHKRRDLPRKPE

SEQ ID NO: 29 - protein, 20 aa, artificial sequence
  Synthetic peptide CADY
GLWRALWRLLRSLWRLLWRA

SEQ ID NO: 30 - protein, 24 aa, artificial sequence
  Synthetic peptide Histatin 5
DSHAKRHHGYKRKFHEKHHSHRGY

SEQ ID NO: 31 - protein, 16 aa, artificial sequence
  Synthetic peptide Cyt86-101
KKKEERADLIAYLKKA

SEQ ID NO: 32 - protein, 30 aa, artificial sequence
  Synthetic peptide
VKKKKIKAEIKIETVTLLVALKVRYRERIT

SEQ ID NO: 33 - protein, 34 aa, artificial sequence
  Synthetic peptide
VKKKKIKAEIKIPSSKSTEIKWKSGKDLTKRSSQ

(19) United States
(12) Patent Application Publication
(10) Pub. No.: US 2018/0066241 A1
(43) Pub. Date: Mar. 8, 2018

(54) PRO-APOPTOTIC SET AND PP2A PEPTIDES

(71) Applicants: Universite Pierre Et Marie Curie (Paris 6), Paris (FR); Institut National De La Sante Et De La Recherche Medicale, Paris (FR); Consejo Superior De Investigaciones Cientificas, Madrid (ES); Katholieke Universiteit Leuven, Leuven (BE)

(72) Inventors: Angelita Rebollo Garcia, Paris (FR); Jeronimo Bravo Sicilia, Valencia (ES); Jesus Maria Fominaya Gutierrez, Madrid (ES); Etienne Waelkens, Rotselaar (BE); Veerle Janssens, Holsbeek (BE)

(21) Appl. No.: 15/556,229

(22) PCT Filed: Mar. 31, 2016
(86) PCT No.: PCT/EP2016/057146

§ 371 Date: Sep. 6, 2017

(30) Foreign Application Priority Data
Mar. 31, 2015 (EP) 15305480.4

(51) Int. Cl. C12N 9/16; C07K 14/47; C07K 7/06; C07K 7/08

(52) U.S. Cl. CPC C12N 9/16; C07K 14/4702; C12Y 301/03016; A61K 38/00; C07K 7/08; C07K 2319/10; C07K 2319/70; C07K 7/06

(57) ABSTRACT

The invention provide chimeric peptides comprising a chimeric peptide comprising a cell-penetrating peptide linked to a pro-apoptotic peptide, wherein the pro-apoptotic peptide is derived from, or consists of, a portion of PP2A protein that binds a SET protein or is derived from, or consists of, a portion of the SET protein that binds PP2A protein.

The invention relates to pro-apoptotic peptides useful in the treatment of a hyperproliferative disease such as tumor, and to chimeric peptides comprising a cell penetrating peptide linked to a pro-apoptotic peptide, wherein the pro-apoptotic peptide binds SET or PP2A proteins.

### BACKGROUND OF THE INVENTION

Protein phosphatase 2A (PP2A) belongs to the serine-threonine phosphatase family that reverse the actions of protein kinases by cleaving phosphate from serine and threonine residues of proteins. PP2A regulates various cellular processes, including protein synthesis, cellular signaling, cell cycle determination, apoptosis, metabolism, and stress responses. Over the past decades, an emerging role of phosphatases in the pathogenesis of tumors has been established. In particular, numerous studies have shown that inhibition of PP2A expression and/or function may contribute to leukemogenesis in several hematological malignancies and thus the tumor suppressing function of PP2A makes it a possible target in anticancer therapy.

The SET protein, also known as I2PP2A, belongs to a family of multitasking proteins, which are involved in apoptosis, transcription, nucleosome assembly, and histone binding. The phosphorylated SET localizes to the nucleus and cytoplasm and has a critical role in the regulation of normal and cancerous signal transduction. It was originally identified as a translocated gene in acute undifferentiated leukemia and elevated expression of SET has been linked to cell growth and transformation. SET which forms a protein complex with PP2A is known as a potent inhibitor of PP2A activity.

Taken together, it is known that SET oncoprotein participates in cancer progression by affecting multiple cellular processes and inhibiting the tumor suppressor PP2A. Therefore, the pharmacological targeting of PP2A/SET complex is likely to represent a valuable approach for the treatment of cancers.

### SUMMARY OF THE INVENTION

The inventors have mapped binding site of SET to PP2Ac and vice-versa. They have then designed peptides showing pro-apoptotic properties, and chimeric peptides wherein a cell penetrating peptide is linked to such pro-apoptotic peptide. In particular, the peptides described herein are useful to disturb the SET/PP2A interaction.

The invention thus provides a chimeric peptide comprising a cell penetrating peptide linked to a pro-apoptotic peptide, wherein the pro-apoptotic peptide is derived from or consists of a portion of PP2A or SET that binds a SET or PP2A protein respectively.

The invention further provides such pro-apoptotic peptide, in particular a pro-apoptotic peptide which derives from or consists of SEQ ID NO: 1 and 2, and a proteolysis-resistant peptide deriving from said pro-apoptotic peptide by one or more chemical modifications, or a substantially homologous peptide, preferably deriving from SEQ ID NO: 1 and 2 by one or more conservative substitutions.

Another subject of the invention is a nucleic acid that encodes the chimeric peptide or the pro-apoptotic peptide as defined herein. A further subject of the invention is a vector comprising said nucleic acid, which is preferably an adenovirus or a lentivirus vector.

The invention also encompasses the peptides, nucleic acid or vector as a medicament (drug).

In particular, the chimeric peptide, the pro-apoptotic peptide, nucleic acid or vector are useful in treating a hyperproliferative disease, preferably a tumor in a patient.

In a particular embodiment, the patient is to be administered with a combination of a chimeric peptide or pro-apoptotic peptide which binds SET, with a chimeric peptide construct or pro-apoptotic peptide which binds PP2A or with a combination of a peptide and a chemotherapeutic drug.

### LEGEND TO THE FIGURES

FIG. 1A and 1B show determination of the binding site of PP2A to SET and vice versa. A) Overlapping dodecapeptides with two amino acid shift covering the whole PP2A protein were bound to a solid support. The membrane was incubated sequentially with SET protein, and anti-SET antibody, followed by a peroxidase-labeled secondary antibody. The membrane was revealed with ECL system. The sequence corresponding to the identified spots is shown. B) Overlapping dodecapeptides with two amino acid shift covering the whole human SET protein were bound to a solid support. The membrane was incubated sequentially with PP2A protein, and anti-PP2A antibody, followed by a peroxidase-labeled secondary antibody. The membrane was revealed with ECL system. The sequence corresponding to the identified spots is shown.

FIG. 2 shows effect of peptide Mut3-DPT-PP2A on apoptosis. H1975 and H1299 cell lines were cultured in the presence or in the absence (control) of the Mut3DPT-PP2Ac peptide at a concentration of 100 μM for 24 h. Apoptosis was detected by Annexin V-FITC staining and analyzed by flow cytometry. Mut3-DPT-PP2A is VKKKKIKAEIKI-ETVTLLVALKVRYRERIT (SEQ ID NO: 32).

FIG. 3 shows effect of peptide Mut3-DPT-SET on apoptosis. MDA-MB231 cell line was cultured in the presence or in the absence (control) of the Mut3DPT-SET peptide at a concentration of 100 μM for 24 h. Apoptosis was detected by Annexin V-FITC staining and analyzed by flow cytometry. Mut3-DPT-SET is VKKKKIKAEIKI-PSSKSTEIKWKSGKDLTKRSSQ (SEQ ID NO: 33).

FIG. 4 shows effect of peptide Mut3-DPT-PP2A on apoptosis. Daudi, Raji and Jok 1 cell lines were cultured in the presence or in the absence (control) of the Mut3DPT-PP2A peptide at a concentration of 100 μM for 24 h. Apoptosis was detected by Annexin V-FITC staining and analyzed by flow cytometry. Mut3-DPT-PP2A is VKKKKIKAEIKI-ETVTLLVALKVRYRERIT (SEQ ID NO: 32).

### DETAILED DESCRIPTION OF THE INVENTION

Definitions

The term “subject” as used herein includes all members of the animal kingdom including non-human primates and humans, preferably male, female, adult, children in need of a treatment wherein a pro-apoptotic effect is desired.

As used herein, the term “treatment” or “therapy” includes, curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. Prophylactic treatment refers to any of: halting the onset, reducing the risk of development, reducing the incidence, delaying the onset, reducing the development, as well as increasing the time to onset of symptoms of a particular disorder.

Two amino acid sequences are “homologous”, “substantially homologous” or “substantially similar” when one or more amino acid residues are replaced by a biologically similar residue or when greater than 80% of the amino acids are identical, or greater than about 90%, preferably greater than about 95%, are similar (functionally identical).

Preferably, the similar or homologous sequences are identified by alignment using, for example, the GCG (Genetics Computer Group, Program Manual for the GCG Package, Version 7, Madison, Wis.) pileup program, or any of the programs known in the art (BLAST, FASTA, etc.). Preferably, these homologous peptides do not include two cysteine residues, so that cyclization is prevented. Preferably the homologous sequences differ by mutations, such as substitutions, insertions and/or deletions of one or several amino acids. Preferably the homologous sequences differ only by conservative substitution(s).

The term “conservative substitution” as used herein denotes the replacement of an amino acid residue by another, without altering the overall conformation and function of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Neutral hydrophilic amino acids, which can be substituted for one another, include asparagine, glutamine, serine and threonine.

By “substituted” or “modified” the present invention includes those amino acids that have been altered or modified from naturally occurring amino acids.

As such, it should be understood that in the context of the present invention, a conservative substitution is recognized in the art as a substitution of one amino acid for another amino acid that has similar properties. Examples of conservative substitutions are set out in the Table 1 below:

TABLE 1
conservative Substitution I

| SIDE CHAIN | |
|---|---|
| CHARACTERISTIC | AMINO ACID |
| Non-polar | G A P I L V |
| Polar uncharged | C S T M N Q |
| Polar-charged | D E K R |
| Aromatic | H F W Y |
| Other | N Q D E |

Alternatively, conservative amino acids can be grouped as described in Lehninger, as set out in Table 2 below.

TABLE 2
conservative substitutions II

| SIDE CHAIN | |
|---|---|
| CHARACTERISTIC | AMINO ACID |
| Non-polar (hydrophobic) | |
| A- Aliphatic | A L I V P |
| B- Aromatic | F W |
| C- Sulfur containing | M |
| D- Bordeline | G |
| Uncharged-polar | |
| A- hydroxyl | S T Y |
| B- Amides | N Q |
| C- Sulfhydryl | C |
| D- Bordeline | G |
| Positively Charged (Basic) | K R H |
| Negatively Charged (Acidic) | D E |

As still another alternative, exemplary conservative substitutions are set out in Table 3, below:

TABLE 3
conservative substitutions III

| Original Residue | Exemplary Substitution |
|---|---|
| Ala (A) | Val (V), Leu (L), Ile (I) |
| Arg (R) | Lys (K), Gln (Q), Asn (N) |
| Asn (N) | Gln (Q), His (H), Lys (K), Arg (R) |
| Asp (D) | Glu (E) |
| Cys (C) | Ser (S) |
| Gln (Q) | Asn (N) |
| Glu (E) | Asp (D) |
| His (H) | Asn (N), Gln (Q), Lys (K), Arg (R) |
| Ile (I) | Leu (L), Val (V), Met (M), Ala (A), Phe (F) |
| Lou (L) | Ile (I), Val (V), Met (M), Ala (A), Phe (F) |
| Lys (K) | Arg (R), Gln (Q), Asn (N) |
| Met (M) | Leu (L), Phe (F), Ile (I) |
| Phe (F) | Leu (L), Val (V), Ile (I), Ala (A) |
| Pro (P) | Gly (G) |
| Ser (S) | Thr (T) |
| Thr (T) | Ser (S) |
| Trp (W) | Tyr (T) |
| Tyr (Y) | Trp (W), Phe (F), Thr (T), Ser (S) |
| Val (V) | Ile (I), Leu (L), Met (M), Phe (F), Ala (A) |

The term “cell-penetrating sequence” (CPP), also named “shuttle peptide” refers to a peptide sequence which facilitates, enhances or increase the transmembrane or intracellular delivery of a peptide into a cell. CPP is able to translocate into cells without causing substantial membrane damage, and can be used as a vector of other molecules when linked to them. The terms refer to cationic cell penetrating peptides, also called transport peptides, carrier peptides, or peptide transduction domains. The CPP, as shown herein, have the capability of inducing cell penetration of a peptide fused to the CPP within 30%, 40%, 50%, 60%, 70%, 80%, 90%, or 100% of cells of a given cell culture population, including all integers in between, and allow macromolecular translocation within multiple tissues in vivo upon systemic administration. A cell-penetrating peptide may also refer to a peptide which, when brought into contact with a cell under appropriate conditions, passes from the external environment in the intracellular environment, including the cytoplasm, organelles such as mitochondria, or the nucleus of the cell, in conditions significantly greater than passive diffusion. This property may be assessed by various methods known by the skilled person. Cell-Penetrating Peptides (CPPs) are also known as protein transduction domains (PTDs), membrane translocating sequences (MTSs), or Trojan peptides.

Pro-Apoptotic Peptide

The pro-apoptotic peptide of the invention induces apoptosis, and is useful for inhibiting cell proliferation, in particular for treating hyperproliferative diseases, such as tumor.

The Pro-apoptotic peptide of the invention is derived from a fragment of PP2A or SET protein that binds a SET or PP2A protein respectively. Said pro-apoptotic peptide is capable of disrupting the interaction between SET and PP2A protein.

A sequence that derives from” or “is derived from” a reference sequence is a peptide sequence that is longer than the reference sequence, or is a homologous sequence, as defined herein.

More particularly, said pro-apoptotic peptide is a portion of the PP2A or SET protein capable of binding SET or PP2A protein respectively, preferably human SET or PP2A. Alternatively, since PP2A and SET protein are well conserved, fragments originating from other animal species can be used, e.g. mouse or rat PP2A or SET protein.

According to one embodiment, the pro-apoptotic peptide binds SET protein. Said pro-apoptotic peptide comprises or consists of ETVTLLVALKVRYRERIT (SEQ ID NO: 1). The peptide of

SEQ ID NO: 1 corresponds to position 95 to 112 of the human Protein phosphatase catalytic subunit, alpha isoform (PPP2CA, also known as PP2Ac or PP2A) by reference to GenBank: CAG33698.1. In another embodiment, the pro-apoptotic peptide binds PP2A. In a preferred embodiment, said pro-apoptotic peptide comprises or consist of PSSKSTEIKWKSGKDLTKRSSQ (SEQ ID NO: 2).Any SET protein isoform may be used, especially isoform 2. Human isoform 2 SET protein is disclosed as NP_003002.2 (NCBI reference Sequence). In the context of the present invention, SET and SET2 may be used interchangeably.

Said pro-apoptotic peptide comprises or consists of amino acid sequence SEQ ID NO: 1 or 2 or a proteolysis-resistant peptide deriving therefrom by one or more chemical modifications. Indeed, certain chemical modifications, in particular N-terminal glycosylation, have been shown to increase the stability of peptides in human serum (Powell et al. (1993), MacEwan SR et al. (2013)). Peptide derivatives also include those with increased membrane permeability obtained by N-myristoylation (Brand, et al . (1996)).

Because some variability may arise from the genomic data from which these peptides derive, and also to take into account the possibility to substitute some of the amino acids present in these peptides without significant loss of apoptotic activity, the invention encompasses peptide derived from said pro-apoptotic peptide by one or more conservative substitutions and substantially homologous peptide, preferably deriving from SEQ ID NO: 1 or SEQ ID NO: 2 by one or more conservative substitutions. The invention also encompasses peptide derived from SEQ IDNO: 1 or SEQ ID NO: 2 by a N and/or C-terminal deletion of 1, 2, 3 or 4 amino acids, preferably of 1 or 2 amino acids.

Such proteolysis-resistant or homologous peptides induce cell apoptosis, in vitro and/or in vivo. Assays for determining whether a molecule, for instance a peptide, induces cell apoptosis are well-known in the art and include, for instance, incubating cells with the candidate peptide and determining if apoptosis is induced by said candidate peptide, e. g. Annexin V and DAPI or PI labelling of cells and identifying as apoptotic cells, those being Annexin V⁺ and DAPI⁻ or PI⁻. Other methods for determining whether a molecule induces cell apoptosis involve following DNA fragmentation by endonuclease or caspase activations.

In a preferred embodiment, said pro-apoptotic peptide of the invention is a peptide of less than 100 amino acid, more preferably 70, 65, 60, 55, 50 or 45 amino acids, preferably less than 40, 35 or 30 amino acids. In some preferred embodiments, said pro-apoptotic peptide is less than 25 to 20 amino acids, said pro-apoptotic peptide is of 18 to 50 amino acid residues, more preferably, 18 to 35 amino acid residues.

Chimeric Peptide

In another embodiment, the present invention is a chimeric peptide comprising an amino acid sequence fused to the N-terminal or the C-terminal end(s) of the pro-apoptotic peptide as described above. The chimeric peptide of the invention induces cell apoptosis.

The pro-apoptotic peptide is fused to one or more amino acid sequence including sequence which allow purification, detection, immobilization, and/or which increases the affinity for SET or PP2A, the bioavailability, the production in expression system and/or stability of said protein.

Said amino acid sequences may be selected from a cell-penetrating peptide, a labeling sequence such as fluorescent protein (e.g. GFP, YFP, BFP), a reporter sequence such as an enzyme tag (luciferase, alkaline phosphatase, glutathione-S-transferase (GST), β-galactosidase), a binding sequence such as an epitope tag (polyHis6, Flag, HA, myc), a DNA binding domain, a hormone binding domain.

Cell-Penetrating Peptide

In a preferred embodiment, said chimeric peptide comprises a cell-penetrating peptide which facilitates, enhances or increases the transmembrane or intracellular delivery of the pro-apoptotic peptide into a cell. In particular, said cell-penetrating sequence allows cellular targeting of the pro-apoptotic peptide, preferably addressing the pro-apoptotic to a specific cell type or cell compartment.

For example, a variety of proteins, including the HIV-1 tat transcription factor, Drosophilia Antennapedia transcription factor, as well as the herpes simplex virus VP22 protein have been shown to facilitate transport of proteins into the cells. Further, an arginine-rich peptide (Futaki (2002)), a polylysine peptide containing Tat PTD (Hashida, et al. (2004)), Pep-1 (Deshayes, et al. (2004)) or an HSP70 protein or fragment thereof (WO 00/31113) is suitable for enhancing intracellular delivery of a peptide or peptidomimetic of the invention into the cell.

In a particular embodiment, the pro-apoptotic peptide may be linked to two, three or more cell-penetrating peptides. Preferably, cell penetrating peptide is a short peptide, of less than about 40 amino acids. Several CPPs can be designed as described in Gautam et al, 2013, incorporated herein by reference.

Preferably, the cell penetrating peptide comprises or consists of:

a) X1-KKKIK-Ψ-EI-X2-X3 (SEQ ID NO: 3); wherein X₁ is vacant, is a lysine residue, or valine-lysine; X₂ is vacant, is a lysine residue, or lysine-isoleucine; X₃ is vacant or is an amino acid sequence of one to 4 amino acids; and Ψ is any amino-acid; or a proteolysis-resistant peptide deriving from SEQ ID NO: 3 by one or more chemical modifications, or a substantially homologous peptide, especially peptides deriving from SEQ ID NO: 3 by one or more conservative substitutions.

b)

(SEQ ID NO: 4)
(RQKRLI)3,
(SEQ ID NO: 5)
(RHSRIG)3,
(SEQ ID NO: 6)
RHSRIGIIQQRRTRNG,
(SEQ ID NO: 7)
RHSRIGVTRQRRARNG,
(SEQ ID NO: 8)
RRRRRRRSRGRRRTY,
or
homologous peptides;

c) Tat peptide, polyarginines peptide, HA2-R9 peptide, Penetratin peptide (Antennapedia), transportan peptide, Vectocell® peptide, maurocalcine peptide, decalysine peptide, HIV-Tat derived PTD4 peptide, Hepatitis B virus Translocation Motif (PTM) peptide, mPrP1-28 peptide, POD, pVEC, EB1, Rath, CADY, Histatin 5, Antp peptide, Cyt86-101 peptide.

In an embodiment, in the cell penetrating peptide of a), X3 is vacant, i.e. the cell penetrating peptide is X₁-KKKIK-Ψ-EI-X₂.

In another embodiment, in the cell penetrating peptide of a), X₁ is VK, X₂ is KI and X₃ is vacant, i.e. the cell penetrating peptide is VKKKKIK-Ψ-EIKI.Preferably Ψ is arginine, lysine, asparagine, or alanine.

The cell-penetrating peptide can thus be VKKKKIKREIKI (SEQ ID NO: 9), VKKKKIKAEIKI (SEQ ID NO:10), VKKKKIKKEIKI (SEQ ID NO: 11) or VKKKKIKNEIKI (SEQ ID NO: 12).

Examples of cell penetrating peptides are shown in Table 4.

TABLE 4
Examples of cell-penetrating peptides

| | | |
|---|---|---|
| Tat peptide | RKKRRQRRR | SEQ ID NO: 13 |
| | YGRKKRRQRRR | SEQ ID NO: 14 |
| polyarginine peptide | R₉ | SEQ ID NO: 15 |
| | R₁₁ | SEQ ID NO: 16 |
| HA2-R9 peptide | GLFEAIEGFIENGWEGMIDGWYG-R9 | SEQ ID NO: 17 |
| Penetratin peptide | RQIKIWFQNRRMKWKK | SEQ ID NO: 18 |
| Transportan peptide | GWTLNSAGYLLGKINLKALAALAKKIL | SEQ ID NO: 19 |
| Maurocalcine peptide | GDCLPHLKLCKENKDCCSKKCKRRGTNIEKRCR | SEQ ID NO: 20 |
| decalysine peptide | KKKKKKKKKK | SEQ ID NO: 21 |
| HIV-Tat derived PTD4 | YARAAARQARA | SEQ ID NO: 22 |
| peptide | | |
| Hepatitis B virus | PLSSIFSRIGDP | SEQ ID NO: 23 |
| Translocation Motif (PTM) | | |
| peptide | | |
| mPrP1-28 peptide | MANLGYWLLALFVTMWTDVGLCKKRPKP | SEQ ID NO: 24 |
| POD peptide | GGG(ARKKAAKA)4 | SEQ ID NO: 25 |
| pVEC peptide | LLIILRRRRIRKQAHAHSK | SEQ ID NO: 26 |
| EB1 peptide | LIRLWSHLIHIWFQNRRLKWKKK | SEQ ID NO: 27 |
| Rath peptide | TPWWRLWTKWHHKRRDLPRKPE | SEQ ID NO: 28 |
| CADY peptide | GLWRALWRLLRSLWRLLWRA | SEQ ID NO: 29 |
| Histatin 5 peptide | DSHAKRHHGYKRKFHEKHHSHRGY | SEQ ID NO: 30 |
| Cyt86-101 peptide | KKKEERADLIAYLKKA | SEQ ID NO: 31 |

Said cell-penetrating peptides may also be a polyarginines peptide which consists of at least 9 arginines or a “Vectocell® peptide” which originates from human heparin binding proteins and/or anti-DNA antibodies.

In a preferred embodiment, the chimeric peptide according to the invention induces cell apoptosis. Said chimeric peptide may preferably have a length comprised between 20 to 100 amino acids, preferably 25 to 80, more preferably 25 to 40 amino acids.

In a preferred embodiment, the chimeric peptide is selected from the group consisting of:

(SEQ ID NO: 32)
VKKKKIKAEIKI-ETVTLLVALKVRYRERIT, this peptide
being designated Mut3-DPT-PP2A;
(SEQ ID NO: 33)
VKKKKIKAEIKI-PSSKSTEIKWKSGKDLTKRSSQ, this peptide
being designated Mut3-DPT-SET;

or homologous or proteolysis peptides deriving thereof.

Further Protection Against Proteolysis:

The N- and C-termini of the pro-apoptotic peptides or chimeric peptides described herein may be optionally protected against proteolysis. For instance, the N-terminus may be in the form of an acetyl group, and/or the C-terminus may be in the form of an amide group.

Internal modifications of the peptides to be resistant to proteolysis are also envisioned, e.g. wherein at least a -CONH12 peptide bond is modified and replaced by a (CH2NH) reduced bond, a (NHCO) retroinverso bond, a (CH2—O) methylene-oxy bond, a (CH2—S) thiomethylene bond, a (CH2CH2) carba bond, a (CO—CH2) cetomethylene bond, a (CHOH—CH2) hydroxyethylene bond, a (N—N) bound, a E-alcene bond or also a —CH═CH—bond.

For instance, the peptide may be modified by acetylation, acylation, amidation, crosslinking, cyclization, disulfide bond formation, formation of covalent cross-links, formation of cysteine, formation of pyroglutamate, formylation, gamma-carboxylation, glycosylation, GPI anchor formation, hydroxylation, iodination, methylation, myristylation, oxidation, phosphorylation, and the like.

The peptides of the invention may be composed of amino acid(s) in D configuration, which render the peptides resistant to proteolysis. They may also be stabilized by intramolecular crosslinking, e.g. by modifying at least two amino acid residues with olefinic side chains, preferably C3-C8 alkenyl chains, preferably penten-2-yl chains) followed by chemical crosslinking of the chains, according to the so-called “staple” technology described in Walensky et al, 2004. For instance, amino acids at position i and i+4 to i+7 can be substituted by non-natural aminoacids that show reactive olefinic residues. All these proteolysis-resistant chemically-modified peptides are encompassed in the present invention.

In another aspect of the invention, peptides are covalently bound to a polyethylene glycol (PEG) molecule by their C-terminal terminus or a lysine residue, notably a PEG of 1500 or 4000 MW, for a decrease in urinary clearance and in therapeutic doses used and for an increase of the half-life in blood plasma. In yet another embodiment, peptide halflife is increased by including the peptide in a biodegradable and biocompatible polymer material for drug delivery system forming microspheres. Polymers and copolymers are, for instance, poly(D,L-lactide-co-glycolide) (PLGA) (as illustrated in US2007/0184015, Hahn SK et al).

Nucleic Acids

The invention also relates to a nucleic acid sequence encoding a peptide according to the invention. The invention further relates to a genetic construct consisting of or comprising a nucleic acid as defined herein, and regulatory sequences (such as a suitable promoter(s), enhancer(s), terminator(s), etc.) allowing the expression (e.g. transcription and translation) of a peptide according to the invention in a host cell. The genetic constructs of the invention may be DNA or RNA, preferably cDNA, and are preferably double-stranded DNA. The polynucleotide of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system). In a preferred but non-limiting aspect, a genetic construct of the invention comprises i)at least one nucleic acid of the invention; operably connected to ii) one or more regulatory elements, such as a promoter and optionally a suitable terminator; and optionally also iii) one or more further elements of genetic constructs such as 3′- or 5′-UTR sequences, leader sequences, selection markers, expression markers/reporter genes, and/or elements that may facilitate or increase (the efficiency of) transformation or integration. In a particular embodiment, the nucleic acid encoding the cell-penetrating peptide of the invention is coupled or fused to a nucleic acid that encodes a peptide or protein of interest. The peptide of interest may be a pro-apoptotic peptide as described herein. More generally, the peptide or protein of interest may be any peptide or protein to express, such as therapeutic peptide or polypeptide, as well as any antigenic or immunogenic peptide if desired. The nucleic acid may especially be carried by a viral vector, such as an adenovirus or a lentivirus, for ex vivo or in vivo infection and expression of the chimeric peptide construct or pro-apoptotic peptide.

Peptide Preparation

Peptides described herein can be synthesized using standard synthetic methods known to those skilled in the art, for example chemical synthesis or genetic recombination. In a preferred embodiment, peptides are obtained by stepwise condensation of amino acid residues, either by condensation of a preformed fragment already containing an amino acid sequence in appropriate order, or by condensation of several fragments previously prepared, while protecting the amino acid functional groups except those involved in peptide bond during condensation. In particular, the peptides can be synthesized according to the method originally described by Merrifield.

Examples of chemical synthesis technologies are solid phase synthesis and liquid phase synthesis. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2- bromobenzyloyycarbonyl), BzI (benzyl), Fmoc (9-fluorenylmcthoxycarbonyl), Mbh (4, 4′- dimethoxydibenzhydryl), Mtr (4-methoxy-2, 3, 6-trimethylbenzenesulphonyl), Trt (trityl), Tos (tosyl), Z (benzyloxycarbonyl) and Clz-BzI (2, 6-dichlorobenzyl) for the amino groups; NO2 (nitro) and Pmc (2,2, 5,7, 8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

Alternatively, the peptide may be synthesized using recombinant techniques. In this case, a nucleic acid and/or a genetic construct comprising or consisting of a nucleotidic sequence encoding a peptide according to the invention, polynucleotides with nucleotidic sequences complementary to one of the above sequences and sequences hybridizing to said polynucleotides under stringent conditions.

The invention further relates to a genetic construct consisting of or comprising a polynucleotide as defined herein, and regulatory sequences (such as a suitable promoter(s), enhancer(s), terminator(s), etc.) allowing the expression (e.g. transcription and translation) of a peptide according to the invention in a host cell.

Thus, in another aspect, the invention relates to a host or host cell that expresses (or that under suitable circumstances is capable of expressing) a peptide of the invention; and/or that contains a polynucleotide of the invention or genetic construct of the invention.

The method of producing the peptide may optionally comprise the steps of purifying said peptide, chemically modifying said peptide, and/or formulating said peptide into a pharmaceutical composition.

Anti-Tumor Therapy

Another aspect of the invention relates to a pro-apoptotic peptide, chimeric peptide, nucleic acid and/or vector as described herein as drug. The drug is useful for increasing cell apoptosis. More particularly, the present invention relates to a pro-apoptotic peptide, chimeric peptide, nucleid acid and/or vector as described herein for use in treating hyperproliferative disease, in particular tumor.

The term “hyperproliferative disorder” refers to disorders characterized by an abnormal or pathological proliferation of cells, for example, tumor, psoriasis, hyperplasia and the like.

The hyperproliferative disease may be a tumor such as haematologic cancer, in particular acute myelogenous leukaemia (AML), chronic lymphocytic leukaemia (CLL), multiple myeloma, Hodgkin's disease, non-Hodgkin's lymphoma, B cell lymphoma, cutaneous T cell lymphoma, or a non-haematologic cancer for instance: brain cancer, epidermoid (in particular lung, breast, ovarian), head and neck (squamous cell), bladder, gastric, pancreatic, head, neck, renal, prostate, colorectal, oesophageal or thyroid cancer, and melanoma. Different types of cancers may include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, lymphoma, leukemia, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma, uveal melanoma and breast cancer. More particularly the peptides described herein or nucleic acids that encode said peptides are useful in the treatment of cancers which exhibit increased SET expression or a mutation of a SET or PP2AA gene.

In a preferred embodiment, the tumor may be a lung cancer, such as for example, non-small cell lung cancer (NSCL), breast cancer, AML, or colorectal cancer. In another preferred embodiment, the tumor may be a chronic lymphocytic leukaemia (CLL).

In other terms, the peptide as defined herein, or nucleic acids that encodes said peptides are useful to treat hyperproliferative disease. Thus, the present invention relates to a method of treatment of a hyperproliferative disease in a subject in need thereof, which method comprises administrating said subject with a pro-apoptotic peptide or a chimeric peptide of the invention or a nucleic acid encoding said pro-apoptotic or chimeric peptide, preferably in combination with an anti-tumor agent, surgery and/or radiotherapy.

Anti-tumor agents include chemotherapeutic agents, including kinase inhibitors, and inhibitors of DNA replication such as DNA binding agents, in particular alkylating or intercalating drugs, antimetabolite agents such as DNA polymerase inhibitors or topoisomerase I or II inhibitors or with anti-mitogenic agents such as alkaloids.

Said peptide or nucleic acid according to the present invention or anti-tumor agent may be administered by any convenient route including intravenous, oral, transdermal, subcutaneous, mucosal, intramuscular, intrapulmonary, intranasal, parenteral, rectal, vaginal and topical. Intranasal route is of particular interest. In a preferred embodiment, the peptides (or nucleic acid that encodes said peptide) may be administered by electroporation.

Electroporation, also known as electropermeabilization or electroinjection, is the permeabilization of cell membranes as a consequence of the application of certain short and intense electric fields across the cell membrane, the cells or the tissues.

Pharmaceutical Compositions

The invention also relates to a pharmaceutical composition, comprising a peptide or nucleic acid that encodes said peptide as described above and a pharmaceutical acceptable carrier. The pharmaceutical composition may also include any other active principle, such as in particular an anti-tumor agent, such as those described above.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules. The pharmaceutical composition comprises a therapeutically effective amount of the peptide, nucleic acid of the invention, e.g. sufficient to show benefit to the individual to whom it is administrated. The pharmaceutical effective dose depends upon the composition used, the route of administration, the type of mammal under consideration, concurrent medication and others factors that those skilled in the medical arts will recognize.

The choice of vehicle and the content of active substance in the vehicle are generally determined in accordance with the solubility and chemical properties of the active compound, the particular mode of administration and the provisions to be observed in pharmaceutical practice. The dosing is selected by the skilled person so that a pro-apoptotic effect is achieved, and depends on the route of administration and the dosage form that is used. Total daily dose of peptides (or nucleic acid that encodes said peptide) administered to a subject in single or divided doses may be in amounts, for example, of from about 0.001 to about 100 mg/kg body weight daily and preferably 0.01 to 10 mg/kg/day, more preferably 5 to 25 mg/kg body weight daily. A daily dosage of about 5 mg/kg is still preferred. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

Preferably the peptide construct (or nucleic acid that encodes said peptide) is administered once a day during a period of at least one week, preferably at least two weeks.

In a particular embodiment, the patient is to be administered with a combination of a chimeric peptide or pro-apoptotic peptide which binds SET and with a chimeric peptide or pro-apoptotic peptide which binds PP2A. Simultaneous administration (i.e., at the same time, as a single composition or separate compositions), or sequential administration is encompassed.

It is also provided a kit comprising a container containing a chimeric peptide or pro-apoptotic peptide which binds SET, and a container containing a chimeric peptide construct or pro-apoptotic peptide which binds PP2A.

The invention further provides a pharmaceutical composition comprising a chimeric peptide or pro-apoptotic peptide which binds SET, in combination with a chimeric peptide construct or pro-apoptotic peptide which binds PP2A.

Another aspect of the invention relates to a combined preparation containing a peptide, nucleic acid, vector as described herein and an anti-tumor agent, for the simultaneous, separate or sequential use in the treatment of a hyperproliferative disease in particular a tumor.

Another aspect of the invention is the use of the pro-apoptotic peptide or chimeric peptide of the invention as a research tool, in particular to study SET-PP2A signaling pathway.

Further aspects and advantages of the present invention will be disclosed in the following experimental section, which should be regarded as illustrative and not limiting the scope of the present application.

### EXAMPLES

### Example 1

### Identification of Binding Site of SET to PP2A and Vice-Versa

Material and Methods

### Peptide Synthesis and Sequence

Peptides were synthesized in an automated multiple peptide synthesizer with solid phase procedure and standard Fmoc chemistry. The purity and composition of the peptides were confirmed by reverse phase HPLC, by amino acid analysis and by mass spectrometry analysis. These peptides were used for protein-protein interaction competition studies or cell culture.

### SET2-PP2Ac Binding Assay on Cellulose-Bound Peptides Containing SET-PP2A Sequences

Overlapping peptides covering the whole human PP2Ac or SET sequence were prepared by automated spot synthesis (Abimed, Langerfeld, Germany) onto an amino-derived cellulose membrane, as described (Frank and Overwin, 1996; Gausepohl et al. 1992). The membranes were blocked, incubated with purified recombinant SET or PP2A protein, after several washing steps, incubated with anti-SET2 (Bethyl Laboratories) or anti-PP2Ac antibody (Santa Cruz), and followed by PO-conjugated secondary antibody. Protein interactions were visualized using ECL system (Pierce).

Results

To identify peptides containing human PP2Ac sequences able to bind to SET, the whole sequence of PP2Ac was synthesized as series of dodecapeptides that were bound to a nitrocellulose support.

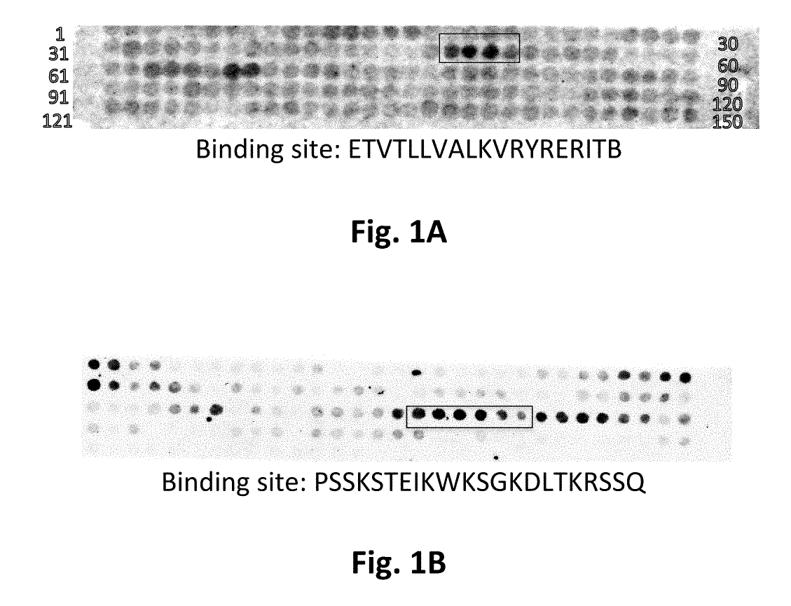

The inventors identified one overlapping sequence of four dodecapeptides proteins. A sequence of 18 amino acid residues that corresponds to the PP2AC sequence bound by SET protein (FIG. 1A). The sequence is: ETVTLLVALKVRYRERIT (SEQ ID NO: 1).

Similarly, to identify peptides containing SET sequences able to bind to PP2Ac, the whole SET sequence was synthesized as series of dodecapeptides that were bound to a nitrocellulose support. The inventors identified one overlapping sequence of six dodecapeptides proteins. A sequence of 22 amino acid residues that corresponds to the SET sequence bound by PP2Ac protein (FIG. 1B). The sequence is PSSKSTEIKWKSGKDLTKRSSQ (SEQ ID NO: 2).

### Example 2

### Design and Effect of Mut3DPT-PP2Ac and Mut3DPT-SET

Material and Methods

Cells

Human non small cell lung carcinoma cell lines H1975 and H1299, as well as breast cancer cell line MDA-MB231 were cultured in DMEM (Gibco Life technologies) medium supplemented with 10% FCS.

Detection of Apoptosis by Annexin-V-FITC Staining

Apopotic cells were detected using Annexin-V (-FITC from BD biosciences) as described by the manufacturer. Briefly, the cells were washed in lx binding buffer, centrifugated and then resuspended in 200 μL of 1× binding buffer containing Annexin V-FITC and propidium iodide (PI). After incubation at room temperature in the dark for 10 min, cells were analyzed with FACS Calibur cytofluometer (BD Biosciences).

The effect of peptides treatment was compared to untreated cells. The peptides were used at 100 μM for 24 h.

Results

The inventors chemically synthesized the two cell-penetrating peptides composed of a shuttle, Mut3DPT-Sh1 (VKKKKIKAEIKI, SEQ ID NO:10) associated to the binding site of PP2Ac to SET and vice versa, then analyzed the capacity of these peptides to induce apoptosis in human non small lung carcinoma cell lines H1975 and H1299, as well as breast cancer cell line MDA-MB231.

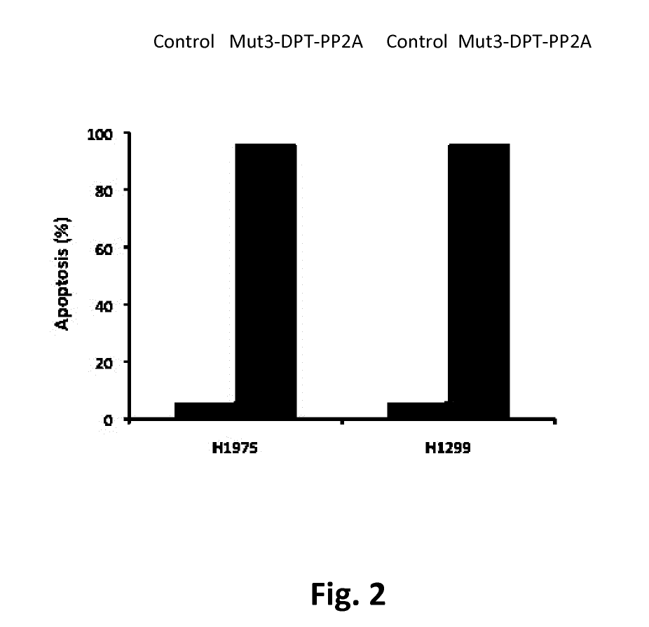

FIG. 2 shows that Mut3DPT-PP2Ac peptide has apoptotic effect in H1975 and 129 cell lines compared to control non treated cells when the peptides were used at 100μM for 24 h.

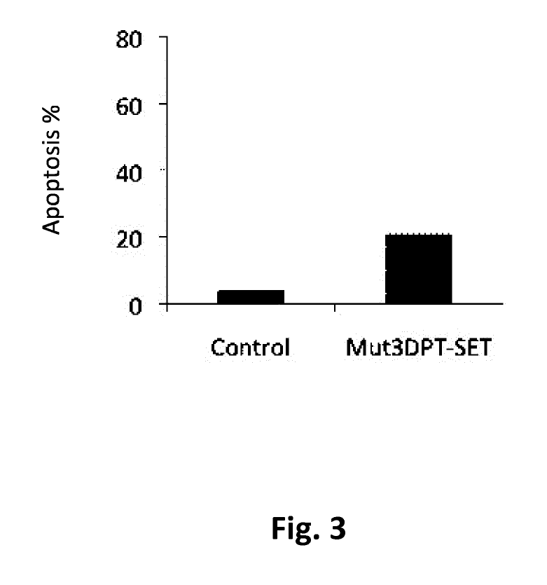

Similar results were obtained in three independent experiments. As illustrated in FIG. 3, Mut3DPT-SET also induces slight apoptosis increase compared to control non treated MDA-MB231 cells.

### Example 3

### Effect of Mut3DPT-PP2Ac and Mut3DPT-SET

Material and Methods

Cells

Human B lymphoblast Daudi cell line, human lymphoblast-like Raji cell line and a Hairy cell leukemia-derived cell line Jok1 were cultured in DMEM (Gibco Life technologies) medium supplemented with 10% FCS.

Detection of Apoptosis by Annexin-V-FITC Staining

Apopotic cells were detected using Annexin-V (-FITC from BD biosciences) as described above.

The effect of Mut3DPT-PP2Ac peptide treatment was compared to untreated cells. The peptides were used at 100 μM for 24 h.

Results

The inventors analyzed the capacity of Mut3DPT-PP2Ac peptide to induce apoptosis in human Daudi, Raji and Jok1 cell lines.

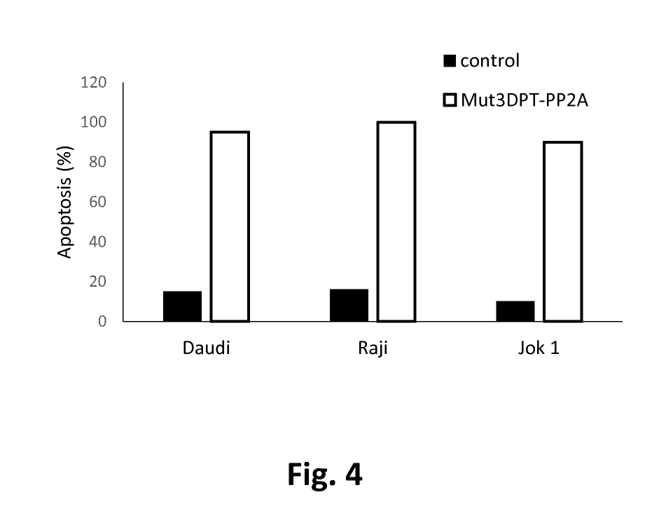

FIG. 4 shows that Mut3DPT-PP2Ac peptide has an apoptotic effect in Daudi, Raji and Jok1 cell lines compared to control non treated cells when the peptides were used at 100μM for 24 h.

### REFERENCES

Brand S H, Holtzman E J, Scher D A, Ausiello D A, Stow J L. Role of myristoylation in membrane attachment and function of G alpha i-3 on Golgi membranes. Am J Physiol. 1996 May;270(5 Pt 1):C1362-9.
Deshayes S, Plénat T, Aldrian-Herrada G, Divita G, Le Grimellec C, Heitz F. Primary amphipathic cell-penetrating peptides: structural requirements and interactions with model membranes. Biochemistry. 2004 Jun. 22;43(24):7698-706.
Frank R, Overwin H. SPOT synthesis. Epitope analysis with arrays of synthetic peptides prepared on cellulose membranes. Methods Mol Biol. 1996;66:149-69.
Futaki S. Arginine-rich peptides: potential for intracellular delivery of macromolecules and the mystery of the translocation mechanisms. Int J Pharm. 2002 Oct. 1;245(1-2):1-7. Review.
Gausepohl H, Boulin C, Kraft M, Frank R W. Automated multiple peptide synthesis. Pept Res. 1992 Nov.-Dec.;5(6):315-20
Gautam A, Chaudhary K, Kumar R, Sharma A, Kapoor P, Tyagi A; Open source drug discovery consortium, Raghava G P. In silico approaches for designing highly effective cell penetrating peptides. J Transl Med. 2013 Mar. 22;11:74. doi: 10.1186/1479-5876-11-74.
Hahn S K, Hoffman A S. Preparation and characterization of biocompatible polyelectrolyte complex multilayer of hyaluronic acid and poly-L-lysine. Int J Biol Macromol. 2005 Dec 30;37(5):227-31. Epub 2006 Jan. 6.
Hashida H, Miyamoto M, Cho Y, Hida Y, Kato K, Kurokawa T, Okushiba S, Kondo S,Dosaka-Akita H, Katoh H. Fusion of HIV-1 Tat protein transduction domain to poly-lysine as a new DNA delivery tool. Br J Cancer. 2004 Mar. 22;90(6):1252-8.
MacEwan S R, Chilkoti A. Harnessing the power of cell-penetrating peptides: activatable carriers for targeting systemic delivery of cancer therapeutics and imaging agents. Wiley Interdiscip Rev Nanomed Nanobiotechnol. 2013;5(1):31-48.
Powell M F, Stewart T, Otvos L Jr, Urge L, Gaeta F C, Sette A, Arrhenius T, Thomson D, Soda K, Colon S M. Peptide stability in drug development. II. Effect of single amino acid substitution and glycosylation on peptide reactivity in human serum. Pharm Res. 1993 Sep.;10(9):1268-73.
Walensky L D, Kung A L, Escher I, Malia T J, Barbuto S, Wright R D, Wagner G,Verdine G L, Korsmeyer S J. Activation of apoptosis in vivo by a hydrocarbon-stapled BH3 helix. Science. 2004 Sep. 3;305(5689):1466-70.

1. A chimeric peptide comprising a cell-penetrating peptide linked to a pro-apoptotic peptide, wherein the pro-apoptotic peptide is derived from, or consists of, a portion of PP2A protein that binds a SET protein or is derived from, or consists of, a portion of the SET protein that binds PP2A protein.

2. The chimeric peptide of claim 1, wherein the pro-apoptotic peptide binds PP2A.

3. The chimeric peptide of claim 2, wherein the pro-apoptotic peptide comprises or consists of : ETVTLLVALKVRYRERIT (SEQ ID NO: 1) or a proteolysis-resistant peptide deriving from said pro-apoptotic peptide by one or more chemical modifications, or a substantially homologous peptide, preferably deriving from SEQ ID NO: 1 by one or more conservative substitutions.

4. The chimeric peptide of claim 1, wherein the pro-apoptotic peptide binds SET.

5. The chimeric peptide of claim 4, wherein the pro-apoptotic peptide comprises or consists of PSSKSTEIKWKSGKDLTKRSSQ (SEQ ID NO:2) or a proteolysis-resistant peptide deriving from said pro-apoptotic peptide by one or more chemical modifications, or a substantially homologous peptide, preferably deriving from SEQ ID NO: 2 by one or more conservative substitutions.

6. The chimeric peptide according to claim 1 wherein cell-penetrating peptide is selected from:
X1-KKKIK-Ψ-EI-X2-X3 (SEQ ID NO: 3), wherein X1 is vacant, is a lysine residue, or valine-lysine;X2 is vacant, is a lysine residue, or lysine-isoleucine; X3 is vacant or is an amino acid sequence of one to 4 amino acids; and Ψ is any amino-acid; or a proteolysis-resistant peptide deriving from SEQ ID NO: 3 by one or more chemical modifications, or a substantially homologous peptide deriving from SEQ ID NO: 3 by one or more conservative substitutions;
(SEQ ID NO: 4)
(RQKRLI)3,
(SEQ ID NO: 5)
(RHSRIG)3,
(SEQ ID NO: 6)
RHSRIGIIQQRRTRNG,
(SEQ ID NO: 7)
RHSRIGVTRQRRARNG,
(SEQ ID NO: 8)
RRRRRRRSRGRRRTY,
or
Tat peptide, polyarginines peptide, HA2-R9 peptide, Penetratin peptide, Transportan peptide, Vectocell peptide, maurocalcine peptide, decalysine peptide, HIV-Tat derived PTD4 peptide, Hepatitis B virus Translocation Motif (PTM) peptide, mPrP1-28 peptide, POD, pVEC, EB1, Rath, CADY, Histatin, Antp peptide, or Cyt86-101peptide.

7. The chimeric peptide of claim 6, wherein said cell-penetrating peptide is X1-KKKIK-Ψ -EI-X2-X3 (SEQ ID NO: 3), wherein Ψ is arginine, alanine, lysine, or asparagines, and X1 is valine-lysine; X2 is lysine-isoleucine; and X3 is vacant.

8. The chimeric peptide according to claim 6, wherein said cell-penetrating peptide is VKKKKIKREIKI (SEQ ID NO: 9), VKKKKIKAEIKI (SEQ ID NO: 10), VKKKKIKKEIKI (SEQ ID NO: 11) or VKKKKIKNEIKI (SEQ ID NO: 12).

9. The chimeric peptide of claim 6 selected from the group consisting of: VKKKKIKAEIKI-ETVTLLVALKVRYRERIT (SEQ ID NO: 32) and VKKKKIKAEIKI-PSSKSTEIKWKSGKDLTKRSSQ (SEQ ID NO: 33).

10. A pro-apoptotic peptide of 18 to 150 amino acid residues wherein said pro-apoptotic peptide comprises or consists of SEQ ID NO: 1 or 2, and a proteolysis-resistant peptide deriving from said pro-apoptotic peptide by one or more chemical modifications, or a substantially homologous peptide, preferably deriving from SEQ ID NO: 1 or 2 by one or more conservative substitutions.

11. A nucleic acid encoding the chimeric peptide as defined in claim 1.

12. A vector comprising the nucleic acid of claim 11.

13. (canceled)

14. A method for treating hyperproliferative disorder, comprising administering an effective amount of the chimeric peptide of claim 1 to a subject in need thereof.

15. (canceled)

16. The method of claim 14, wherein the hyperproliferative disorder is a cancer.

17. A nucleic acid encoding the pro-apoptotic peptide as defined in claim 10.

18. A vector comprising the nucleic acid of claim 17.

19. A method for treating a hyperproliferative disorder, comprising administering an effective amount of the proapoptotic peptide of claim 10 to a subject in need thereof.

20. The method of claim 19, wherein the hyperproliferative disorder is a cancer.
